(19) United States
(12) Patent Application Publication
(10) Pub. No.: US 2008/0044486 A1
(43) Pub. Date: Feb. 21, 2008

(54) Controlled Food Effect Composition

(76) Inventors: Goran Nilsson, Stockholm (SE); Lena Lindman, Stockholm (SE); Peter Kaufmann, Jarna (SE); Karin Bohlinder, Solna (SE); Jan Holmback, Vaxholm (SE)

Correspondence Address: DICKSTEIN SHAPIRO LLP, 1177 AVENUE OF THE AMERICAS (6TH AVENUE), NEW YORK, NY, 10036-2714, US

(21) Appl. No.: 10/580,786

(22) PCT Filed: Nov. 24, 2004
(86) PCT No.: PCT/SE04/01727

§ 371 Date: Aug. 22, 2007

(30) Foreign Application Priority Data
Nov. 25, 2003 (SE) 0303135-8

(51) Int. Cl. A61K 9/14; A61K 31/01; A61K 31/22; A61P 43/00; A61K 36/899

(52) U.S. Cl. 424/502; 424/750; 514/546; 514/547; 514/762

(57) ABSTRACT

The invention provides a pharmaceutical composition for oral administration comprising an active substance having a food effect, in combination with a lipid material comprising membrane lipids, which is characterised in showing a reduced food effect. The invention also refers to the use of a lipid material comprising membrane lipids for the manufacture of a pharmaceutical composition having a reduced food effect.

The present invention refers to a pharmaceutical composition containing an active substance, which composition reduces or eliminates the food effect of said active substance, which substance when taken concomitantly with food will give a change in bioavailability, rate of on-set, duration of therapeutic effect or incidence and seriousness of side effects as compared to when given in a fasted state.

### BACKGROUND

Oral administration of drugs is frequently affected by food-drug interactions, a phenomenon often concluded by the term “food effect”. As generally interpreted, food effect is a very broad term including all aspects of interactions of food on drug dissolution, absorption, distribution, metabolism and elimination, that is the entire pharmacokinetic fate of the drug. The implications of food effect include for instance changes in bioavailability, rate of on-set, duration of therapeutic effect and incidence and seriousness of side effects.

The food effect is an issue of great importance during the development of a drug. If differences in the pharmacokinetic profile after administration in the fasted or fed state are too high, it can be difficult to define a safe and efficient therapeutic window for the drug. The bioavailability may vary unacceptably depending on whether or not the drug has been taken with food, what kind of food has been taken etc. Postprandial drug absorption may also be altered compared to the absorption from a fasting state in a way by which toxic effects can result.

In fact, discovery of food-drug interactions frequently results in that otherwise promising development projects are stopped by the developing company because of this lack of control.

In some cases where food-drug interactions lead to an increase of drug absorption, the drug is recommended to be taken with food in order to be sufficiently absorbed and to exert its expected clinical effect. Examples of products on the market with such recommendations are acitretin, amiodarone, artemether, atovaquone, clofazimine, etretinate, fenofibrate, isotretinoin, itraconazole, lumefantrine, ribavirin, saquinavir and tenofovir disoproxil. It is of course not ideal to develop such drug formulations. Depending on what kind of food has been taken drug absorption can vary. If, by mistake, the patient would forget to take the drug formulation with food, clinical inefficiency could be the result of a too low absorption.

The systemic availability, or bioavailability, of an orally administered drug can in a simplified manner be described as the outcome of the following chain of processes: dissolution, absorption, distribution, metabolism and elimination. Though in literature there are examples of food-drug interactions influencing all of these steps, dissolution and absorption are the most obvious since at these stages the drug is actually in physical contact with the bulk of food. Distribution and metabolism might also be important, perhaps most commonly in an indirect way. The dissolution and absorption stages may direct the uptake to a certain region in the gastrointestinal tract, and subsequently determine the distribution and site of metabolism.

The pharmacokinetic profile of a drug is commonly described by the following parameters: Maximum plasma concentration (Cₘₐₓ), Time to maximum concentration (Tₘₐₓ), half-life (T_{1/2}) and Area under the curve (AUC). These parameters are of course to some extent interdependent, and are influenced to varying degree of the outcome of the above mentioned processes. By way of example, for a certain drug Cₘₐₓ might be mostly influenced by the rate of dissolution and absorption, Tₘₐₓ mostly by dissolution and distribution, T_{1/2} mostly by metabolism and elimination and AUC more or less influenced by all processes.

It is quite obvious that in most cases changes in dissolution and absorption will have a significant impact on all parameters except perhaps on T_{1/ 2}. This implies that a formulation system, by which these two processes can be controlled and be made independent of food intake, will provide a more reliable and safer administration of the drug. Depending on the indication, the pharmacokinetic parameter that is most closely connected to therapeutic effect is either AUC, Cₘₐₓ, Tₘₐₓ or combinations thereof.

In a review (W. N. Charman, Journal of Pharmaceutical Sciences vol. 89, 2000, pp 967-978), a generally reasonable interpretation of postprandial “food effect” bioavailability data was presented with regard to lipid-induced physicochemical (e.g. enhanced wetting and solubilization) and physiological responses (e.g. transit changes, increased intestinal secretions, changed local blood flow). However, in order to use this information for the development of new dosage forms one has to bear in mind the large lipid volume differences between a meal and a pharmaceutically acceptable dosage form.

The effects of food on clinical pharmacokinetics have been reviewed (B. N. Singh, Clinical Pharmacokinetics, vol. 37(3), 213-255, 1999). In this review the effect of formulation type is discussed, where, in bioequivalence studies, under fasting and fed conditions, differences in pharmacokinetic parameters may be attributed to different formulation principles and excipient systems. The predominant role of pharmaceutical formulations applies not only for drug absorption, but also for hepatic first-pass metabolism, that a formulation exhibiting a good dissolution profile is less likely to be affected by a high-fat meal in spite of the lipophilicity of the drug. It is further mentioned that it is believed that the absorption of drugs solubilized by polyglycolised (polyethoxylated) glycerides is not affected by the presence or absence of food in the stomach.

An alternative route to systemic circulation is intestinal lymphatic transport. Highly lipophilic compounds, such as long-chain triglycerides, lipid-soluble vitamins and cholesterol gain access to systemic circulation via the lymphatic system. It has been suggested that for intestinal lymphatic transport to be a signifycantly important contribution to oral bioavailability, candidate drugs need to be highly lipophilic (e.g. LogP>5), reasonably soluble in long-chain triglycerides and administered in the presence of an adequate lipid source.

Particulate uptake in the intestines is a possible route for absorption of drugs that are poorly soluble in water. Unlike classic drug absorption, which is governed by solubility, particulate uptake is more likely governed by factors related to the liquid-solid interface, such as surface tension, wettability, particle size and, for ionisable surfaces, the z potential. Therefore, it can be speculated that the unfavourable solubility properties of a poorly soluble drug molecule can be concealed in a particle system, thus rendering it more amenable to absorption.

Production of solid lipid nanoparticles have been discussed by Müller, et al, European J. Pharm. and Biopharm, 50, 161-177, 2000, examples of a sustained release system for camptothecin, a bioavailability enhancing system for piribedil and an adverse effect reducing system for cyclosporin are given. Mehnert and Mäder, in Adv. Drug Deliv. Rev., 47, 165-196, 2001, state “it can be expected that food will have a large effect on SLN (solid lipid nanoparticles) performance, however no experimental data have been published on this issue to our knowledge.”

While in many cases lipid based formulations have increased the bioavailability of a drug that is poorly absorbed from more conventional preparations, such improved preparations can still show a considerable food effect, or in some cases the food effect can become reversed. As discussed above, food effects commonly lead to unacceptable variability in the efficiency of a therapy, and can also constitute a hurdle for development of otherwise promising drug candidates. Food effect is also likely to present a problem for delivery systems intended to act through particulate uptake. There is therefore a strong need in the pharmaceutical industry today for a drug delivery system that can eliminate or reduce the food effect.

### PRIOR ART

U.S. Pat. No. 6,534,088 B2 discloses an orally administered pharmaceutical composition for the treatment of elevated levels of cholesterol and related conditions comprising a statin and fenofibrate in the form of microparticles of solid fenofibrate that are stabilized by phospholipids as a surface active substance, wherein a therapeutically effective amount of fenofibrate of the composition provides the statin and a quantity of fenofibrate to a fasted human patient that is greater than 80% of the quantity of fenofibrate provided by the same amount of the composition when administered to the same patient who has been fed a high-fat meal.

U.S. Pat. No. 6,338,857 relates to a pharmaceutical composition consisting of a coated tablet giving sustained release of an active substance with the additional feature of being free of food effect. The composition consists of a core comprising the active substance (preferably carbamazepine) and a coating comprising a gastroresistant polymer and a hydrophilic silicon dioxide.

In WO 00/25772 it is stated that to ensure uniform and optimal absorption of drugs administered orally, the surface area of the drug must be as large as possible, one extreme being when the drug is present as separate molecular entities completely dissolved in the GI-fluids. For poorly soluble drugs this is not possible and thus it is necessary to reduce particle or crystal size, by other means, as much as possible, thereby maximizing the surface area of the drug exposed to the GI-fluids and enabling rapid and uniform absorption. The inventors claim that by micronization of isotretinoin to a particle size between 5 and 30 μm, and by decreasing the wax content of the formulation from 22% down to 18.2%, a formulation was produced that gave minimal difference in absorption between fed and fasted state. The conclusion regarding the optimal conditions was however made despite that the differences, both between groups and between fed and fasted state, were non-significant.

WO 95/20943 discloses oil-in-water emulsions based on an oily material and an emulsifier comprising a galactolipid material. The emulsions are suitable for use as carriers for an active substance in a pharmaceutical composition, formulated for oral, parenteral and topical administration among others. The oily material is any lipophilic material having a liquid or semi-solid consistency at room temperature. The emulsions have the advantage of being stable and to exhibit a narrow and consistent particle size.

WO 95/20945 describes lipophilic carriers, having a continuous lipid phase. The lipophilic carriers comprise galactolipids in combination with non-polar lipids and optionally a polar solvent, and are suitable for incorporation of drugs, but also as formulations for skin care, nutrition and food.

WO 00/32219 is related to a new formulation of cyclosporin A, which does not contain any toxic excipients. The composition, comprising cyclosporin A, membrane lipids, monoglycerides and non-polar lipids, was tested in a human bioavailability study on fasted subjects and found to be bioequivalent to the reference preparation Neoral.

Mueller, et al, Pharmaceutical Res., 11, 151-155, 1994, compared the pharmacokinetics of cyclosporin A, as the Sandimmune® formulation, with cyclosporin A as the Sandimmune Neoral® formulation, when given in the fed or fasted state. They found that food, as a high-fat meal, had a pronounced effect on absorption from the Sandimmune formulation (37% increase in AUC), and a less pronounced effect on the Sandimmune Neoral formulation (26% decrease in AUC). They attributed this difference to the fact that in the GI-fluids, Sandimmune produced a crude oil-in-water emulsion while Sandimmune Neoral produced a fine microemulsion.

### DEFINITIONS

“Food effect”, as used herein, refers to a relative difference of at least 20% in AUC (Area under the curve), Cₘₐₓ (Maximum plasma concentration), and/or Tₘₐₓ (Time to maximum concentration) of an active substance, when said substance or a formulation thereof, such as a tablet or a capsule, is administered orally to a mammal, preferably a human, concomitantly with food or in other words in a fed state as compared to when the same formulation is administered in a fasted state. The food effect F is calculated as

F=(Y_{fed}−Y_{fasted})/Y_{fasted}

wherein Y_{fed} and Y_{fasted} are the found values of AUC, Cₘₐₓ or Tₘₐₓ in the fed and fasted state, respectively.

“Positive food effect”, as used herein, refers to a food effect where the AUC and/or Cₘₐₓ is higher when the drug is administered orally in fed state than when it is administered in the fasted state.

“Negative food effect” refers to a food effect where the AUC and/or Cₘₐₓ is lower when the drug is administered orally in fed state than when it is administered in the fasted state. Drug-food interactions leading to reduced incidence and/or severity of side effects are referred to as an “enhanced tolerability food effect”.

“Reduced food effect”, as used herein, refers to the reduction of food effect of a composition of an active substance in comparison with another composition of said active substance. The reduction in food effect is calculated as (|F_{comp}|−|Fᵢₙᵥ|)/″F_{comp}|, where F_{comp} is the food effect of the comparative formulation and Fᵢₙᵥ is the food effect of the formulation according to the invention.

For the purpose of this invention, a meal is defined as an intake of food, containing at least 5 g of fat and giving at least 250 kcal.

“Concomitantly with food” or “administration in a fed state”, as used herein, refers to administration from about 30 minutes before the meal to about 1 hour after the meal.

“Administration in a fasted state”, as used herein, refers to administration at least 4 hours after a meal. Moreover, a fasted state also requires continued fasting for at least 2 hours after the administration.

### DESCRIPTION OF THE INVENTION

The present invention refers to a pharmaceutical composition for oral administration comprising an active substance having a food effect, in combination with a lipid material comprising membrane lipids, characterised in showing a reduced food effect.

Preferably the pharmaceutical composition is an immediate release formulation for oral administration. An immediate release formulation is defined herein as a formulation, which has not been deliberately designed to give a release of the active substance that is delayed or prolonged.

The lipid material comprises membrane lipids and non-polar lipids, and optionally other polar lipids.

The amount of membrane lipids is preferably not less than about 1% by weight of the lipid material, more preferred not less than about 3% by weight of the lipid material, even more preferred not less than about 10% by weight of the lipid material, most preferred from about 20% to about 60% by weight of the lipid material.

Membrane lipids according to the invention are polar lipids occurring naturally in the cell membrane of animals or plants. Membrane lipids are phospholipids, glycolipids and sphingolipids, or mixtures thereof. Examples of phospholipids are phosphatidyl choline, phosphatidyl ethanolamine, phosphatidyl inositol and phosphatidyl glycerol; glycolipids can be exemplified by digalactosyldiacylglycerols monogalactosyldiacylglycerols and sulfoquinovosyldiacylglycerols; and sphingolipids by sphingomyelin, ceramides and cerebrosides.

Membrane lipids should preferably comprise glycolipids, more preferably galactolipids, and most preferred digalactosyldiacylglycerols. The preferred amount of digalactosyldiacylglycerols is not less than about 0.5% by weight of the lipid material, more preferred not less than about 1% by weight of the lipid material, even more preferred not less than about 5% by weight of the lipid material, most preferred from about 10% to about 30% by weight of the lipid material.

It may be advantageous to use a mixture of several different membrane lipids. The membrane lipids can also, entirely or partly, be modified by for example hydrogenation or partial hydrolysis.

The fatty acid composition of the membrane lipids may vary. Preferably the fatty acids, constituting parts of the membrane lipids have an average chain length of 14-20 carbon atoms. Even more preferred the fatty acids, constituting parts of the membrane lipids, have an average chain length of 16-19 carbon atoms. The fatty acid parts, constituting parts of the membrane lipids may be saturated, unsaturated or polyunsaturated. Preferably, the average number of double bonds in the fatty acids, constituting parts of the membrane lipids, is at least 0.6 and not more than 2.5.

Other polar lipids are for instance, semi-synthetic or synthetic analogues to membrane lipids. Polar lipids typically consists of a one or several fatty acid moieties and/or long chain amines, constituting a hydrophobic part, and a hydrophilic part comprising hydroxyl groups, amino groups, phosphates, sulfonates or other polar functional groups. Polar lipids can be nonionic, anionic, cationic or zwitterionic.

The non-polar lipids to be used in the composition of the invention typically are mono-, di- or triacylglycerols or other related compounds, similar in structure or properties, and mixtures thereof. By way of example vegetable oils, animal oils, synthetic oils, fatty acids, natural and synthetic glycerides, sterol esters, fatty alcohols can be mentioned.

Monoglycerides, or monoacylglycerols, are 1-monoacylglycerols or 2-monoacylglycerols or mixtures thereof, the acyl group being a fatty acid moiety of at least 3 carbon atoms. In medium chain monoglycerides, the acyl groups have an average chain length of 6-12, more preferred 8-10 carbons atoms. Long chain monoglycerides have an average chain length of more than 12 carbon atoms.

Preferably the content of monoglycerides is less than about 70% by weight, more preferred from about 2 to about 60%, most preferred from about 5 to about 50% by weight. Monoglycerides preferably comprise at least 50% by weight medium chain monoglycerides, more preferred at least 80%.

It is preferred that at least about 10% by weight, more preferred at least about 20%, of the lipid material comprises di- or triglycerides, or a mixture thereof.

Optionally, additional ingredients may be added to the composition of the invention. Examples of such ingredients include but are not limited to polar solvents.

Polar solvents are for instance water or alcohols with up to 8 carbon atoms and 1-3 hydroxyl groups or other related compounds, similar in structure or properties, and mixtures thereof. Preferred examples are water, ethanol, propylene glycol and glycerol.

According to a preferred embodiment of the invention the lipid material comprises fractionated cereal oil. Fractionated cereal oil refers to a lipid material, rich in membrane lipids, which is obtained by extraction from a vegetable oil. Preferably the vegetable oil is obtained from oats, barley, wheat, rye, rice, maize, sesame, soy, or a mixture of these. More preferably the oil is obtained from oats, barley, wheat or rye. Most preferably the oil is obtained from oats. Fractionated cereal oil obtained from oat oil is referred to as fractionated oat oil herein. The fractionated cereal oil comprises not less than about 20% by weight of membrane lipids, more preferred not less than about 30% by weight of membrane lipids, most preferred not less than about 40% by weight of membrane lipids. Fractionated cereal oil also comprises non-polar lipids. Examples of non-polar lipids in the fractionated cereal oil include but are not limited to triglycerides of fatty acids, diglycerides of fatty acids, monoglycerides of fatty acids and free fatty acids.

A large number of substances exhibit a food effect when administrated orally, and are hence suitable for formulation in accordance with the invention. As an illustration of the diversity of these compounds, Table 1 below shows some examples.

TABLE 1
Examples of active substances showing a food
effect in conventional pharmaceutical compositions.

| Active | Molecular | | | Food effect in a conventional |
|---|---|---|---|---|
| substance | Weight | LogP | Category | composition (references) |
| Acetylsalicylic | 180.2 | 1.2 | Analgesic; antipyretic; anti- | Delayed absorption and reduced |
| acid | | | inflammatory | side effects (1, 2) |
| Acitretin | 326.4 | 6.4 | Antipsoriatic | Increased absorption (2, 4) |
| Amiodarone | 645.3 | 7.8 | Antiarrhythmic (class III) | Increased absorption (3) |
| Artemether | 298.4 | 3.5 | Antimalarial | High-fat food gives increased |
| | | | | absorption (5) |
| Atovaquone | 368.1 | 4.9 | Antipneumocystic | High-fat food gives increased |
| | | | | absorption (1, 3, 4) |
| Clofazimine | 473.4 | 7.7 | Antibacterial (tuberculostatic, | Increased absorption (2, 4) |
| | | | leprostatic) | |
| Cyclosporin A | 1202.6 | 3.2 | Immunosuppressant | High-fat food gives increased |
| | | | | absorption (4) |
| Danazol | 337.5 | 4.3 | Antigonadotropin | Increased absorption (1, 3, 4) |
| Etretinate | 354.5 | 6.6 | Antipsoriatic | High-fat food gives increased |
| | | | | absorption (1, 4) |
| Felodipine | 384.3 | 3.9 | Antihypertensive; antianginal | High-fat food gives increased |
| | | | | absorption (1) |
| Fenofibrate | 360.8 | 5.1 | Antihyperlipoproteinemic | Increased absorption (4) |
| Fenretinide | 391.5 | 6.1 | Antineoplastic | High-fat food gives increased |
| | | | | absorption (3) |
| Griseofulvin | 352.8 | 2.2 | Antifungal | High-fat food gives increased |
| | | | | absorption (1) |
| Halofantrine | 500.4 | 8.8 | Antimalarial | Increased absorption (2) |
| Isotretinoin | 300.4 | 6.3 | Antiacne | Increased absorption (7) |
| Itraconazole | 705.6 | 5.7 | Antifungal | Increased but delayed absorption |
| | | | | (2) |
| Loratadine | 382.9 | 5.2 | Antihistaminic | Increased but delayed absorption |
| | | | | (1, 4) |
| Lumefantrine | 528.9 | 8.9 | Antimalarial | High-fat food gives increased |
| | | | | absorption (4, 5) |
| Manidipine | 611.5 | 4.1 | Antihypertensive | Increased absorption (2) |
| Nifedipine | 346.3 | 2.2 | Antianginal; antihypertensive | High-fat food gives increased |
| | | | | absorption, delayed absorption |
| | | | | and reduced side effects (1, 3) |
| Oltipraz | 226.3 | 1.7 | Anticarcinogen | Increased absorption (6) |
| Raloxifene | 473.6 | 5.5 | Antiosteoporotic | Increased absorption (1) |
| Ribavirin | 244.2 | −1.9 | Antiviral | High-fat food gives increased |
| | | | | absorption (5) |
| Ritonavir | 721.0 | 5.0 | Antiviral | Increased absorption (1, 4, 5) |
| Rufinamide | 238.2 | 0.8 | Anticonvulsant | Increased absorption (2) |
| Saquinavir | 670.9 | 3.8 | Antiviral | Increased absorption (7) |
| Sirolimus | 914.2 | 5.3 | Immunosuppressant | High-fat food gives increased |
| | | | | absorption (4, 5) |
| Spironolactone | 416.6 | 2.8 | Diuretic | Increased absorption and reduced |
| | | | | side effects (1) |
| Tenofovir | 519.4 | 0.6 | Antiviral | High-fat food gives increased |
| disoproxil | | | | absorption (5) |
| Valproic acid | 144.2 | 2.8 | Anticonvulsant; antimanic; | Delayed absorption and reduced |
| | | | antimigraine | side effects (1, 3, 4) |

### References in Table 1.

1. Bland S. E., Drug-Food Interactions, J Pharmacy Soc 1998 Nov/Dec
2. Singh B. N., Effects of food on clinical pharmacokinetics, 1999; 37 (3); 213-55
3. Welling P. G., Effect of food on drug absorption, Pharmac Ther 1989; 4; 424-441
4. Martindale—The Complete Drug Reference, 33^{rd} edition 2002
5. FASS—Swedish pharmacopoeia 2004 http://www.fass.se/LIF/produktfakta/sok_lakemedel.jsp
6. Homeida M. M., Ali H. M., Sulaiman S. M., Bennet J. L., Oltipraz: administration with food increases its anti-schistosomal activity, Trans R Soc Trop Med Hyg. 1986; 80(6); 908-10
7. Schmidt L E, Dalhoff K., Food-drug interactions, Drugs; 2002;62(10);1481-502
   The pharmaceutical composition of the invention can comprise an active substance selected from the group consisting of analgesics, anti-inflammatory agents, anti-helmintic, anti-antallergeic agents, anti-arrythmics, antibacterials, anticoagulants, anticonvulsants, anti-depressants, antidiabetic agents, anti-epileptic agents, antifungals, antigout agents, antihistamines, antihyperlipoproteinemics, antihypertensive agents, antimalarial agents, antimuscarinics, antimycobacterial agents, antineoplastic agents, antiosteoporotics, antiprotozoal agents, antithyroids, antivirals, anxiolytic agents, beta-adrenorecoptor blockers, cardiac inotropic agents, cephalosporines, corticosteroids, cough suppressants, diagnostic agents, diuretics, dopaminergics, enzymes, gastro-intestinal agents, hormones, hypnotics, immunologycal agents, immunosuppressants, antihyperlipid-emics, mucolytics, mucle relaxants, opoid analgesics, parasympathomimics, parathyroid agents, peptides, prostaglandins, retinoids, sedatives, sympathomimetics, vasodilators and vitamins and other nutrients.

The active substance can be selected from the group consisting of acitretin, alendronic acid, amiodarone, aminophylline, artemether, atorvastatin, atovaquone, baclofen, Bay-X-1005, buspirone, cefuroxime, CGP 43371, clofazimine, cyclosporin A, danazone, eletriptane, enalapril, estradiol, etretinate, felodipine, fenofibrate, fenretinide, griseofulvin, halofantrine, isotretionoin, itraconazole, loratadine, lovastatin, lumefantrine, manidipine, metronidazole, nabumetone, nifedipine, oltipraz, pergolide, phenytoin, pidotimod, prednisolone, procainamide, propafenone, propranolol, raloxifene, raloxiphene, ribavirin, ritonavir, rufinamide, saquinavir, simvastatin, sirolimus, spironolactone, tenofovir diso-proxil, theophylline, tramadol, trazodone, triazolam, troglitazone, vanoxerine, warfarin, vinpocetine and zolmitriptan, or salts thereof.

According to the invention the pharmaceutical composition comprises the active substance dissolved or dispersed in a lipid material. It is preferred that the active substance is dissolved in the composition. If the lipid solubility of the active substance is low, it may be advantageous to disperse all or a part of the active substance as solid particles in the composition. If a dispersion of solid particles of the active substance is used, it is advantageous to use a small and well defined particle size. Preferably the mean particle size as measured by laser diffraction is less than about 20 μm, preferably less than about 10 μm, more preferred less than about 5 μm, most preferred less than about 1 μm. This can be achieved by micronisation of the active substance as understood by the person skilled in the art of particle size reduction. A small and well defined particle size may also be achieved by dissolving the active substance in a hot lipid or in a solvent during the manufacturing process. Particles of the active substance may then be formed upon cooling or evaporation. The particle size can be controlled by varying the process parameters as understood by the skilled artisan.

Another embodiment of the invention can be described as a self-emulsifying drug delivery system for oral administration. A self-emulsifying system as referred to herein is a system, which after immersion in water or an aqueous environment, forms an emulsion with a minimum of agitation. A self-emulsifying system according to the invention is characterised by, after addition of about 0.05 g of the formulation into a vial containing 10 ml of aqueous release medium at 37° C., forming an emulsion with a mean particle size of less than 100 μm after 5 minutes of shaking at 250 rpm. The aqueous release medium may be either 0.1 M HCl, phosphate buffer pH 6.8 or distilled water. The tested formulation will be considered a self-emulsifying system if an emulsion is formed in one, two or all of these media. The particle size is preferably determined by laser diffraction. In this embodiment of the invention, the active substance is dissolved or dispersed in a lipid material. Preferably, the self-emulsifying drug delivery system is filled into capsules, as understood by a person skilled in the art of encapsulation. It may be advantageous to use soft capsules made from gelatin or gelatin-free materials such as hydroxypropyl methylcellulose or starch.

In another embodiment of the invention the composition is an emulsion. Preferably, most of the active substance is dispersed or dissolved in oil droplets, comprising membrane lipids, said oil droplets being dispersed in an aqueous medium. It may be advantageous to use the membrane lipids as an emulsifier in such an emulsion. Optionally, additional emulsifiers may be used such as cationic surfactants, anionic surfactants or non-ionic surfactants.

Another embodiment of the invention is a solid lipid particle dispersion, which can be described as a composition where the drug is dissolved or dispersed into a lipid material, which is solid at room temperature and where said lipid material is dispersed in a polar solvent and where the lipid material comprises membrane lipids. Preferably, the lipid material has a melting point or softening point less than about 45° C., more preferred less than about 40° C. and most preferred less than about 37° C. The mean particle diameter of the dispersed solid lipid particles, determined by laser diffraction, is preferably less than about 20 μm, more preferred not less than about 10 μm, and most preferred less than about 5 μm.

The amount of active substance in the composition may vary, depending on factors such as the required dose and the lipid solubility of the active substance. Preferably, the amount of active substance is less than about 50% by weight, more preferred from about 0.001 to about 50% by weight, even more preferred from about 1 to about 40% by weight, most preferred from about 1 to about 25% by weight of the composition.

The present invention provides a pharmaceutical composition in which the food effect of an active substance is reduced or eliminated. Preferably, the food effect is reduced by at least about 25%, preferably 50%, more preferred by at least 75%, even more preferred by at least about 90%.

Even more preferred the food effect is eliminated. An eliminated food effect is by definition obtained when the difference in AUC, Cₘₐₓ and/or Tₘₐₓ, when a formulation is administered orally to a mammal, preferably a human, concomitantly with food compared to when the same formulation is administered in the fasted state, is less than about 20%, more preferred less than about 10%.

Cyclosporin A, isotretinoin and many antivirals are examples of drugs that show an increased absorption when taken with food. Especially cyclosporin A has been subject for extensive formulation work, and pharmacists have indeed succeeded in developing a product, Neoral® (Sandimmune Neoral®), a lipid based solution of cyclosporin A, which gives a higher bioavailability and show a less pronounced food effect than the original preparation Sandimmune®.

A preferred embodiment of the invention is a pharmaceutical composition for oral administration of isotretinoin comprising from 0.5 to about 12% by weight of isotretinoin, preferably from about 1 to about 8% by weight of isotretinoin most preferred from about 1 to about 2% by weight of isotretinoin. The composition also comprises from about 30% to 99.5% by weight of a lipid material in which the isotretinoin is dissolved or dispersed. The lipid material comprises from about 2% to about 60% by weight of membrane lipids, preferably from about 3% to about 50% by weight, and from about 40% to about 98% by weight of non-polar lipids. Preferably, the lipid material comprises fractionated cereal oil. The pharmaceutical composition may be formulated as a liquid or semisolid material filled in capsules or as an oil-in-water emulsion.

Another preferred embodiment of the invention is a pharmaceutical composition for oral administration of an immunosuppressant comprising from 0.1 to about 20% by weight of immunosuppressant, preferably from about 1 to about 10% by weight of immunosuppressant. This composition additionally comprises from about 1% to about 40% by weight of membrane lipids, preferably from about 15% to about 25% by weight of membrane lipids, where the immunosuppressant is dissolved or dispersed in a mixture comprising said membrane lipids and other polar and non-polar lipids. Preferably, the composition comprises fractionated cereal oil. Preferably the composition comprises from about 5% to about 40% by weight of medium chain monoglycerides. The pharmaceutical composition may be formulated as a liquid or semisolid material filled in capsules or as an oil-in-water emulsion. Examples of immunosuppressants include but are not limited to azathioprine, cyclosporin A, mercaptopurine, mycophenolic acid, pidotimod, sirolimus and tacrolimus, and derivatives and salts thereof. Preferably the immunosuppressant is cyclosporin A.

Another preferred embodiment of the invention is a pharmaceutical composition for oral administration comprising up to about 50% by weight of an antiviral, preferably from about 5 to about 40% by weight, more preferred from about 10 to about 30% by weight, most preferred from about 15 to about 25% by weight of an antiviral. Examples of antivirals include but are not limited to nucleosides such as aciclovir, famciclovir, ganciclovir, ribavirin and valaciclovir; protease inhibitors such as indinavir, nelfinavir, ritonavir and saquinavir; nuclesoside analogues such as abacavir, didanosine, lamivudinine, stavudinine, zalcitabine, zidovudine, or salts of any of these. Preferably the antiviral is a protease inhibitor. More preferably, the antiviral is indinavir, nelfinavir, ritonavir or saquinavir, or salts thereof. The composition of this embodiment preferably comprises from about 10% to about 70% by weight of membrane lipids, preferably from about 20 to about 60% by weight, and most preferred from about 30 to about 50% by weight of membrane lipids. Preferably, the membrane lipids are provided by fractionated cereal oil. Additionally, the composition of this embodiment may comprise from about 10% to about 70% by weight of medium chain monoglycerides, preferably from about 20% to about 60% by weight, and most preferred from about 30 to about 50% by weight of medium chain monoglycerides.

### EXAMPLES OF FORMULATIONS

Ingredients used in the formulation examples:

Fractionated oat oil, Type 1 (LipoCore Holding AB, Sweden) obtained by ethanol extraction of oat oil resulting in a mixture comprising approximately 51% by weight of non-polar lipids, mainly di- and triglycerides (approximately 48% by weight of the total composition), and approximately 49% by weight of membrane lipids such as galactolipids and phospholipids. The content of digalactosyldiacylglycerols is approximately 21% by weight of the total composition.

Fractionated oat oil, Type 2 (LipoCore Holding AB, Sweden), obtained by ethanol extraction of oat oil resulting in a mixture comprising approximately 58% by weight of non-polar lipids, mainly di- and triglycerides (approximately 55% by weight of the total composition), and approximately 42% by weight of membrane lipids such as galactolipids and phospholipids. The content of digalactosyldiacylglycerols is approximately 19% by weight of the total composition.

CPL Galactolipids (LipoCore Holding AB, Sweden), obtained by extraction and purification of oat oil, with a content of digalactosyldiacylglycerols of not less than 40% by weight, the remainder being other membrane lipids.

Akoline MCM (Karlshamns AB, Sweden), a mixture of capric and caprylic monoacylglycerols (60%), diacylglycerols (32%) and triacylglycerols (8%)

Medium chain monoglycerides (LipoCore Holding AB, Sweden), obtained by fractionation of Akoline MCM resulting in capric and caprylic monoacylglycerols (98% combined)

Medium chain triglycerides (LipoCore Holding AB, Sweden), obtained by fractionation of a commercial MCT oil, Acomed R (Karlshamns AB, Sweden), resulting in more than 99.5% triacylglycerols, mostly of capric and caprylic acids.

Acosoft 36 (Karlshamns AB, Sweden), hydrogenated coco-glycerides (Ph.Eur.)

Evening primrose oil (LipoCore Holding AB, Sweden), purified triglycerides from evening primrose (>99%).

Palm oil (LipoCore Holding AB, Sweden), fractionated triglycerides from palm oil (>99.5%).

### Example 1

### Isotretinoin Formulation 1 (IT-1)

| Ingredient | % by weight |
|---|---|
| Isotretinoin | 1.0 |
| Fractionated oat oil | 49.5 |
| Akoline MCM | 49.5 |

2.0 g isotretinoin, 99 g Akoline MCM and 99 g Fractionated oat oil, Type 2, were weighed into a 500 ml round-bottomed flask. 50 ml of 95% ethanol was added, the flask was put in a Rotavapor (Buchi, Switzerland) heated to 60° C. and stirred at 100 rpm for 15 minutes until the ingredients were mixed and the isotretinoin crystals were dissolved. The mixture was then evaporated to complete dryness at 50 mbar and 60° C., for 15 minutes. 200 g of final product, a yellow-brown, homogenous viscous matrix was obtained. The mixture was filled into hard gelatin capsules (Coni-Snap size 00, Capsugel), 0.75 g per capsule.

### Example 2

### Isotretinoin Formulation 2 (IT-2)

| Ingredient | % by weight |
|---|---|
| Isotretinoin | 1.0 |
| Fractionated oat oil | 3.0 |
| Akosoft 36 | 40.0 |
| Water | 56.0 |

2.72 g isotretinoin, 109.1 g Akosoft 36 and 8.18 g Fractionated oat oil, Type 2, were weighed into a beaker and preheated to 67° C. The mixture was then added to 140 ml of water, preheated to 66° C., under high-shear mixing for 5 min at 13000 rpm and another 5 min at 17000 rpm. The mixture was homogenized (Rannie homogenizer; Model MINI-LAB, Type 8.30H; APV Rannie AS, Denmark) at 600 bar and approximately 60° C. for five cycles. 250 g of a white fine emulsion was obtained. The average particle diameter was determined by laser diffraction in a Mastersizer 2000 (Malvern, UK) to <2 μm. The emulsion was filled into 10 ml brownish coloured oral syringes (BD Medical System) 6 g/syringe.

### Example 3

### Isotretinoin Formulation 3 (IT-3)

| Ingredient | % by weight |
|---|---|
| Isotretinoin | 8 |
| CPL Galactolipids | 46 |
| Akosoft 36 | 36.8 |
| Akoline MCM | 9.2 |

8.0 g isotretinoin, 9.2 g Akoline MCM, 36.8 g Akosoft 36 and 46 g CPL Galactolipids were weighed into a 500 ml round-bottomed flask. 50 ml of 95% ethanol was added, the flask was put in a Rotavapor (Buchi, Switzerland) heated to 60° C. and stirred at 100 rpm for 15 minutes until the ingredients were mixed and the isotretinoin crystals were dissolved. The mixture was then evaporated to complete dryness at 50 mbar and 60° C., for 15 minutes. 100 g of final product, a yellow-brown, highly viscous suspension was obtained. The mixture was filled into hard gelatin capsules, Coni-Snap size 00, Capsugel, 0.75 g per capsule.

### Example 4

### Cyclosporin A Formulation 1 (CS-1)

| Ingredient | % by weight |
|---|---|
| Cyclosporin A | 10 |
| Fractionated oat oil | 45 |
| Akoline MCM | 45 |

24.76 g of Fractionated oat oil, Type 1, was weighed into a 100 ml bottle together with 24.75 g of Akoline MCM and 5.49 g of cyclosporin A. The mixture was agitated by means of a magnetic stirrer for approximately 20 minutes at 60° C. until the ingredients were mixed and the cyclosporin A crystals were dissolved. A yellow-brown, highly viscous liquid was obtained. The mixture was filled into hard gelatin capsules (Coni-Snap size 00, Capsugel), 0.75 g per capsule.

### Example 5

### Cyclosporin A Comparative Formulation (CS-2)

| Ingredient | % by weight |
|---|---|
| Cyclosporin A | 10 |
| CPL Galactolipids | 18 |
| medium chain monoglycerides | 72 |

9.90 g of CPL Galactolipids was weighed into a 100 ml bottle together with 39.60 g of Medium chain monoglycerides and 5.50 g of cyclosporin A. The mixture was agitated by means of a magnetic stirrer for approximately 2 hours at 60° C. and then overnight at 30° C. until the ingredients were mixed and the cyclosporin A crystals were dissolved. A yellow-brown, highly viscous liquid was obtained. The mixture was filled into hard gelatin capsules (Coni-Snap size 00, Capsugel), 0.75 g per capsule.

### Example 6

### Acetylsalicylic Acid Formulation

| Ingredient | % by weight |
|---|---|
| Acetylsalicylic acid | 30 |
| CPL Galactolipids | 35 |
| Palm oil | 28 |
| Acoline MCM | 7 |

50 g of CPL Galactolipids was weighed into a 250 ml round-bottomed flask together with 40 g of palm oil, 10 g of Acoline MCM. 50 ml of 95% ethanol was added and the mixture was stirred for 5 min at 70° C. until the ingredients were mixed. The mixture was then evaporated to complete dryness at 70° C., for 35 minutes. 14 g of the mixture was weighed into a 50 ml beaker together with 6 g of acetylsalicylic acid (Ph.Eur.). The mixture was agitated by means of a magnetic stirrer at 60° C. until the ingredients were mixed. A white solid suspension was obtained. The mixture was filled into hard gelatin capsules (Coni-Snap size 00, Capsugel), 0.75 g per capsule.

### Example 7

### Ritonavir Formulation

| Ingredient | % by weight |
|---|---|
| Ritonavir | 20 |
| Fractionated oat oil | 40 |
| Medium chain monoglycerides | 40 |

14 g of Fractionated oat oil, Type 2, is weighed into a 250 ml round-bottomed flask together with 14 g of Medium chain monoglycerides and 7 g of ritonavir. 40 ml of 95% ethanol is added and the mixture is stirred for 10 min at 70° C. until the ingredients are mixed and the saquinavir crystals are dissolved. The mixture is then evaporated to complete dryness at 70° C. 35 g of final product, a yellow-brown medium viscous liquid is obtained. The mixture is filled in hard gelatin capsules (Coni-Snap size 00, Capsugel), 0.75 g per capsule.

### Example 8

### Sirolimus Formulation

| Ingredient | % by weight |
|---|---|
| Sirolimus | 0.1 |
| Fractionated oat oil | 2.9 |
| Akoline MCM | 10 |
| Medium Chain Triglycerides | 87 |

0.1 g sirolimus, 10 g Akoline MCM, 2.9 g Fractionated oat oil, Type 2, and 87 g Medium chain triglycerides are weighed into a 250 ml bottle. The mixture is agitated by means of a magnetic stirrer at 60° C. until the ingredients are mixed and the sirolimus crystals are dissolved. A clear liquid is obtained. The mixture is filled into hard gelatin capsules (Coni-Snap size 00, Capsugel), 0.75 g per capsule. Reference preparations used in the bioavailability studies:

Neoral® Soft Gelatin Capsules 100 mg (Novartis) Cyclosporin A 100 mg, ethanol 9.5% (w/v), corn oil mono-, di- and triglycerides, polyoxyl 40 hydrogenated castor oil, dl-α-tocopherol, and propylene glycol filled in soft gelatin capsules (gelatin, glycerol and dyes).

Accutane® Capsules 20 mg (Roche Pharmaceuticals) Isotretinoin (13-cis-retinoic acid) 20 mg, beeswax, butylated hydroxyanisole, edetate disodium, hydrogenated soybean oil flakes, hydrogenated vegetable oil and soybean oil filled in soft gelatin capsules (gelatin, glycerol and dyes).

### Bioavailability Studies

In investigations of food-drug interactions it is important to define the characteristics and timing of the meals. A common procedure in the studies is to, after over-night fasting, administer the test preparation in the morning with or without concomitant intake of a standardized breakfast. Other schemes and types of standardized meals are of course possible.

The standardized meal can have different levels of fat, protein, fiber and other potentially interacting components. By way of example, a high-fat breakfast can consist of 60 g white bread, 30 g sausage, one egg, 30 g cheese, 20 g butter, 175 g yoghurt (7% fat) and 300 ml of apple juice. This example will give 966 kcal, 56 g of fat, 26 g of protein and 3 g of fiber. A low-fat breakfast may contain as little as about 5 grams of fat giving only about 250 kcal.

### Isotretinoin Bioavailability in Humans

For this study, formulations IT-1, IT-2 and IT-3 were evaluated and compared with the commercially available preparation Accutan (IT-Ref).

The chemical, physical and microbiological stability of the formulations was monitored for three months and found without any adverse remarks.

Eighteen healthy men (age 18-50) were after information and written consent entering the study. Physical examinations including medical histories and routine laboratory tests were performed at the entry (visit no 1). Subjects were excluded if any deviation of clinical relevance was discovered during the screening examination. Other criteria for exclusion were, e.g. smokers, BMI>28 and migraine.

This was an open, crossover design study. The subjects were divided into three groups, six in each group. Each group received reference formulation with and without food and a test formulation with and without food. The amount given was a single dose of 60 mg isotretinoin on each occasion. There were in total three different test formulations. A standardized breakfast was given 15 minutes prior to the drug formulation in the cases of food intake. Blood was sampled from an intravenous indwelling catheter in an arm vein before administration and at 0.5, 1, 2, 3, 4, 6, 8, 11, 24, 48, and 72 hours after drug administration.

Blood samples were collected in covered 4.5 ml EDTA tubes. Within 1 hour, plasma was obtained by centrifugation 10 min at 3000 g and 4° C., thereafter the plasma was frozen in dark colored cryo vials at −70° C. (Teerlink et al.) The samples were stored nine to twelve months prior to analytical assay, the last three months storage was at −20° C.

Sample preparation and HPLC conditions were as follows. The plasma was acidified and proteins precipitated with acetonitrile. Samples were diluted with water prior to injection onto a C-18 (250×4.6 mm) column equilibrated with 85% mobile phase A containing 40 mmol/l ammonium acetate buffer pE 5.75 and methanol 50:50, (v/v) and 15% mobile phase B, 100% methanol, at 30° C. Flow rate was 0.8 m/min and the gradient was from 15% to 100% B in 22 minutes. Isotretinoin was detected at 340 nm and quantified on the basis of peak area using external calibration. The samples were analysed one subject at the time, including test preparations with and without food and reference (IT-Ref) with and without food.

The analytical assay of isotretinoin in plasma was evaluated according to Guidance for Industry (FDA). The accuracy and precision of the method were monitored continuously using quality control plasma pools spiked with isotretinoin at three different levels. Aliquots of these were stored at −20° C. and analysed, two at each level, every analytical run.

Maximum plasma concentration (Cₘₐₓ) and the area under the curve (AUC) were calculated from the concentration data. AUC was estimated by the linear trapezoidal rule, that is small area segments were summarized.

The pharmacokinetical data for all three formulations and references respectively are summarized in Table 2.

TABLE 2
Pharmacokinetic parameters for different
preparations of isotretinoin, administered with or without
food. Average values and standard error of mean.

| Treatment | Cₘₐₓ, rel. | AUC, rel. |
|---|---|---|
| IT-ref, fasted | 100 | 100 |
| IT-ref, with food | 230 ± 16 | 288 ± 16 |
| IT-ref, food effect | +130 | 30 188 |
| IT-1, fasted | 505 ± 51 | 327 ± 31 |
| IT-1, with food | 202 ± 21 | 276 ± 34 |
| IT-1, food effect | −60 | −16 |
| IT-2, fasted | 449 ± 39 | 343 ± 35 |
| IT-2, with food | 299 ± 25 | 342 ± 48 |
| IT-2, food effect | −33 | −0 |
| IT-3, fasted | 162 ± 54 | 156 ± 47 |
| IT-3, with food | 309 ± 43 | 313 ± 65 |
| IT-3, food effect | +91 | +100 |

In the interpretation of the results from this study, it was anticipated that the parameter most likely to reflect the clinical effect of isotretinoin is AUC. Furthermore, it can be assumed that the incidence and seriousness of some side effects are more related to Cₘₐₓ.

The results presented in Table 4 show that for IT-1, Cₘₐₓ in the fasted state is considerably higher even compared to IT-ref given with food, but when IT-1 is given with food Cₘₐₓ is much lower. However, AUC for IT-1 in the fasted state is comparable to that for IT-ref given with food, and moreover, almost unchanged when given with food. In other words, in terms of Cₘₐₓ the food effect has been reduced by 54% and reversed. In terms of AUC the food effect has been reduced by 91%, or almost eliminated. For IT-2 the food effect has been reduced by 75% and reversed in terms of Cₘₐₓ, and eliminated in terms of AUC. IT-3 gives a slightly higher absorption (both Cₘₐₓ and AUC) than IT-ref and the food effect has been reduced by 30% for Cₘₐₓ and 47% for AUC.

Hence the three test compositions all give an increased absorption and a reduced or eliminated food effect compared to IT-ref (Accutane).

### Cyclosporin A Bioavailability in Humans

This study was designed to study the influence of concomitant food intake and administration on the bioavailability of Cyclosporin A. Two different formulations were used in this study, CS-1 and CS-2.

The commercially available preparation of Cyclosporin A Neoral® was used as a reference, CS-Ref. According to the Swedish Pharmacopoeia CS-Ref experiences an up to 26% decrease in Cₘₐₓ and a 15% decrease in AUC when taken with food.

In a similar study design as for isotretinoin, CS-1, CS-Comp and CS-Ref were given with and without a standardized breakfast. The number of healthy volunteers in each group was 6, and there were two treatment groups. The dose was 300 mg.able 3. Pharmacokinetic parameters obtained after administration of different cyclosporin A formulations with or without food.

| Treatment | Cₘₐₓ, relative | AUC, relative |
|---|---|---|
| CS-ref, fasted | 100 | 100 |
| CS-ref, with food | 98 | 84 |
| CS-ref, food effect | −2 | −16 |
| CS-1, fasted | 101 | 84 |
| CS-1, with food | 80 | 84 |
| CS-1, food effect | −21 | 0 |
| CS-2, fasted | 44 | 38 |
| CS-2, with food | 52 | 58 |
| CS-2, food effect | +20 | +53 |

In the case of cyclosporin A, the parameter considered to be most relevant from a clinical point of view is AUC.

The results in Table 3 confirm earlier reports that CS-ref (Neoral) exhibits a moderate negative food effect, that is AUC decreases when CS-ref is given with food. It is also notable that formulation CS-1 in terms of AUC is not susceptible to any food effect. On the other hand the absorption of formulation CS-2 is suppressed when given without food but increases when given with food. Thus, the food effect on CS-2 is the opposite of that observed for CS-Ref.

CS-ref (Neoral) has been recognized as a truly successful example of formulation art and the results from this study confirms that this preparation only shows a moderate negative food effect. However, CS-ref contains polyethoxylated surfactants, which potentially could cause adverse reactions. In contrast, CS-1, which is essentially bioequivalent with CS-ref in terms of bioavailability and absence of food effect, contains only natural food chain lipids, and could therefore be expected to be better tolerated.

### CONCLUSIONS

The bioavailability studies show that by using different types of pharmaceutical compositions of isotretinoin and cyclosporin A, comprising membrane lipids and non-polar lipids, formulations are obtained which reduce or eliminate the food effect.

1. A pharmaceutical composition for oral administration comprising an active substance having a food effect, in combination with a reduced food effect effective amount of a lipid material comprising membrane lipids.

2. The pharmaceutical composition according to claim 1, wherein the content of membrane lipids is not less than 3% by weight of the lipid material.

3. The pharmaceutical composition according to claim 1, wherein the membrane lipids contain digalactosyl- diacylglycerol in an amount not less than about 0.5% by weight of the lipid material.

4. The pharmaceutical composition according to claim 1, wherein the lipid material comprises a fractionated cereal oil.

5. The pharmaceutical composition according to claim 1, wherein the lipid material comprises non-polar lipids.

6. The pharmaceutical composition according to claim 1, wherein the composition comprises the active substance dissolved or dispersed in solid lipid particles with a diameter of not more than 20 pm.

7. A pharmaceutical composition for oral administration according to claim 1 comprising from 0.5 to 12% by weight of the composition of isotretinoin, and a lipid material comprising from 2% to 60% by weight of membrane lipids, and from 30 to 98% by weight of non-polar lipids, calculated on the lipid material.

8. A pharmaceutical composition for oral administration according to claim 1, comprising from 0.1 to 20% by weight of an immunosuppressant, from 1% to 40% by weight of membrane lipids, and from 5 to 40% by weight of monoglycerides, calculated on the composition.

9. A pharmaceutical composition for oral administration according to claim 1, comprising up to about 50% by weight of an antiviral, from about 10% to about 70% by weight of membrane lipids, and from about 10% to about 70% by weight of monoglycerides, calculated on the composition.

10. The pharmaceutical composition according to claim 1, wherein the lipid material comprises about 3% to about 60% by weight of monoglycerides.

11. The pharmaceutical composition according to claim 1, wherein the monoglycerides comprise medium chain monoglycerides.

12. The pharmaceutical composition according to claim 1, wherein the lipid material comprises at least 10% by weight of di-and triglycerides or a mixture thereof.

13. The pharmaceutical composition according to claim 1 comprising in addition a polar solvent.

14. A pharmaceutical composition comprising solid lipid particles with a diameter of not more than about 20 um, comprising a) an active substance, dissolved or dispersed in said lipid particles, and b) a lipid material comprising membrane lipids.

15. The pharmaceutical composition according to claim 1, wherein the reduced food effect effective amount is sufficient to provide a reduction of at least 25%.

16. The pharmaceutical composition according to s, having a claim 1, wherein the food effect is less than 20%.

17. (canceled)

18. The pharmaceutical composition according to claim 2, wherein the lipid material comprises at least about 20% by weight of fractionated oats oil.

19. The pharmaceutical composition according to claim 1, wherein the membrane lipids contain digalactosyl-diacylglycerol in an amount not less than about 0.5% by weight of the lipid material, and the lipid material comprises non-polar lipids.

20. The pharmaceutical composition claim 19, wherein the active substance is dissolved or dispersed in solid lipid particles with a diameter of not more than 20 pm.

21. The pharmaceutical composition according to claim 20, wherein the lipid material comprises about 3% to about 60% by weight of monoglycerides, and at least 10% by weight of di-and triglycerides or a mixture thereof.
